(19) United States
(12) Patent Application Publication
(10) Pub. No.: US 2022/0008115 A1
(43) Pub. Date: Jan. 13, 2022

(54) ELECTRODE UNIT AND ENDOSCOPE SYSTEM

(71) Applicant: OLYMPUS CORPORATION, Tokyo (JP)

(72) Inventors: Soichi IKUMA, Tokyo (JP); Tsuyoshi HAYASHIDA, Tokyo (JP)

(73) Assignee: OLYMPUS CORPORATION, Tokyo (JP)

(21) Appl. No.: 17/385,221

(22) Filed: Jul. 26, 2021

Related U.S. Application Data
(63) Continuation of application No. PCT/JP2019/002750, filed on Jan. 28, 2019.

(51) Int. Cl. A61B 18/14

(52) U.S. Cl. CPC A61B 18/14; A61B 2018/00083

(57) ABSTRACT

An electrode unit comprises: an electrode supporting member provided with a pair of distal end rigid members surfaces of which are covered by an electrically insulating material; a treatment electrode configured with electrode bodies arranged in respective distal end portions of the pair of distal end rigid members and protruding downward respectively from the pair of distal end rigid members, and a spanning portion that spans respective lower end portions of the electrode bodies; and a detection sensor arranged on respective proximal end sides of the pair of distal end rigid members and configured to detect a contact state between the pair of distal end rigid members and tissue.

### CROSS REFERENCE TO RELATED APPLICATION

This application is a continuation application of PCT/JP2019/002750 filed on Jan. 28, 2019, the entire contents of which are incorporated herein by this reference.

### BACKGROUND OF THE INVENTION

### 1. Field of the Invention

The present invention relates to an electrode unit and an endoscope system configured to treat tissue inside a subject by using a high-frequency current.

### 2. Description of the Related Art

Electrocautery has been known as a technique of treating (for example, resecting or coagulating) tissue of a subject such as a human body. For example, Japanese Patent No. 3730796 discloses a device configured to treat (for example, to resect or to coagulate) tissue inside a subject by using a high-frequency current under observation with an endoscope. The technique disclosed in Japanese Patent No. 3730796 carries out treatment (for example, resection or coagulation) of tissue by passing a high-frequency current through an electrode formed in a loop shape.

The electrode formed in a loop shape as disclosed in Japanese Patent No. 3730796 is used for, for example, resecting tissue in an organ such as the bladder. Here, the depth to which the electrode is thrust into a wall surface of the organ varies depending on the strength of force with which a user presses the electrode against the wall surface.

### SUMMARY OF THE INVENTION

An electrode unit according to one aspect of the present invention is configured to resect or coagulate tissue inside a subject by using a high-frequency current, and includes: an electrode supporting member provided with a pair of distal end rigid members surfaces of which are covered by an electrically insulating material; a treatment electrode configured with electrode bodies arranged in respective distal end portions of the pair of distal end rigid members and protruding downward respectively from the pair of distal end rigid members, and a spanning portion that spans respective lower end portions of the electrode bodies; and a detection sensor arranged on respective proximal end sides of the pair of distal end rigid members and configured to detect a contact state between the pair of distal end rigid members and the tissue.

An electrode unit according to another aspect of the present invention is configured to resect or coagulate tissue inside a subject by using a high-frequency current, and includes: an electrode supporting member provided with a pair of distal end rigid members surfaces of which are covered by an electrically insulating material, and an elastic member rigidity of which is lower than rigidity of the pair of distal end rigid members; a treatment electrode configured with electrode bodies arranged in respective distal end portions of the pair of distal end rigid members and protruding downward respectively from the pair of distal end rigid members, and a spanning portion that spans respective lower end portions of the electrode bodies; a proximal end rigid member arranged on a proximal end side of the elastic member; a bend detection sensor configured to detect a bend amount of the elastic member; and an attitude detection sensor configured to detect an inclination amount with respect to a gravity direction of the pair of distal end rigid members.

An electrode unit according to yet another aspect of the present invention is configured to treat tissue inside a subject by using a high-frequency current under observation with an endoscope, and includes: an electrode supporting member inserted into the subject, an external surface of which is configured with an electrically insulating material; a distal end rigid member provided in a distal end portion of the electrode supporting member; a treatment electrode supported by the distal end rigid member and protruding from an external surface of the distal end rigid member; a collection electrode electrically connected with the treatment electrode; and a detection electrode arranged on a proximal end side relative to the treatment electrode on a face of the external surface of the distal end rigid member, the face being oriented in a protrusion direction of the treatment electrode, the detection electrode being configured to detect a contact state between the distal end rigid member and the tissue.

An endoscope system according to one aspect of the present invention includes: the electrode unit according to the one aspect of the present invention; a high-frequency power control device configured to control output of high-frequency waves; and a resistance detection circuit provided in the high-frequency power control device and configured to detect a resistance value of a current flowing in the electrode unit, wherein in the high-frequency power device, the detection sensor detects a resistance value of a current flowing between the pair of distal end rigid members, and an output of a high-frequency current to the treatment electrode is controlled on the basis of the resistance value.

An endoscope system according to another aspect of the present invention includes: the electrode unit according to the other aspect of the present invention; a high-frequency power control device configured to control output of high-frequency waves; and a resistance detection circuit provided in the high-frequency power control device and configured to detect a resistance value of a current flowing in the electrode unit, wherein in the high-frequency power device, the detection electrode detects a resistance value of a current flowing between the pair of distal end rigid members, and an output of a high-frequency current to the treatment electrode is controlled on the basis of the resistance value.

An endoscope system according to yet another aspect of the present invention includes: the electrode unit according to the other aspect of the present invention; a high-frequency power control device configured to control output of high-frequency waves; and a resistance detection circuit provided in the high-frequency power control device and configured to detect a resistance value of a current flowing in the electrode unit, wherein in the high-frequency power device, the detection electrode detects a resistance value of a current flowing between the pair of distal end rigid members, and an output of a high-frequency current to the treatment electrode is controlled on the basis of the resistance value.

### BRIEF DESCRIPTION OF THE DRAWINGS

FIG. 1 is a diagram showing a schematic configuration of an endoscope system according to a first embodiment;

FIG. 2 is a diagram showing an electrode unit according to the first embodiment seen along a first axis;

FIG. 3 is a diagram showing the electrode unit according to the first embodiment seen along a second axis;

FIG. 4 is a cross-sectional view taken along a line IV-IV in FIG. 3;

FIG. 5 is a cross-sectional view taken along a line V-V in FIG. 4;

FIG. 6 is a cross-sectional view taken along a line VI-VI in FIG. 4;

FIG. 7 is a flowchart showing an operation of the high-frequency power control device of the first embodiment;

FIG. 8 is a diagram showing a manner of resecting tissue by using the electrode unit according to the first embodiment;

FIG. 9 is a diagram showing a manner of resecting tissue by using the electrode unit according to the first embodiment;

FIG. 10 is a diagram showing a manner of resecting tissue by using the electrode unit according to the first embodiment;

FIG. 11 is a diagram showing a schematic configuration of an endoscope system according to a second embodiment;

FIG. 12 is a flowchart showing an operation of the high-frequency power control device of the second embodiment;

FIG. 13 is a diagram showing a schematic configuration of an endoscope system according to a third embodiment;

FIG. 14 is a cross-sectional view showing a configuration of a detection electrode of the third embodiment;

FIG. 15 is a flowchart showing an operation of the high-frequency power control device of the third embodiment;

FIG. 16 is a diagram showing a schematic configuration of an endoscope system according to a fourth embodiment;

FIG. 17 is a flowchart showing an operation of the high-frequency power control device of the fourth embodiment;

FIG. 18 is a diagram showing an electrode unit according to a fifth embodiment seen along the first axis;

FIG. 19 is a flowchart showing an operation of the high-frequency power control device of the fifth embodiment;

FIG. 20 is a diagram showing a manner of resecting tissue by using the electrode unit according to the fifth embodiment;

FIG. 21 is a diagram showing a manner of resecting tissue by using the electrode unit according to the fifth embodiment;

FIG. 22 is a diagram showing the electrode unit according to a sixth embodiment seen from above along the second axis;

FIG. 23 is a diagram showing an electrode unit according to the sixth embodiment seen along the first axis;

FIG. 24 is a diagram showing a state in which the elastic region of the electrode unit according to the sixth embodiment is bent; and

FIG. 25 is a diagram showing a fourth index of the sixth embodiment.

### DETAILED DESCRIPTION OF THE PREFERRED EMBODIMENTS

Hereinafter, preferred embodiments of the present invention are described with reference to the drawings. Note that, in each of the drawings used for the following description, constitutive elements may have different scales in order that each of the constitutive elements may have a recognizable size on the drawings, and the present invention is not limited to the number, shapes, ratios of size, and relative positional relationships of the constitutive elements featured in these drawings.

### First Embodiment

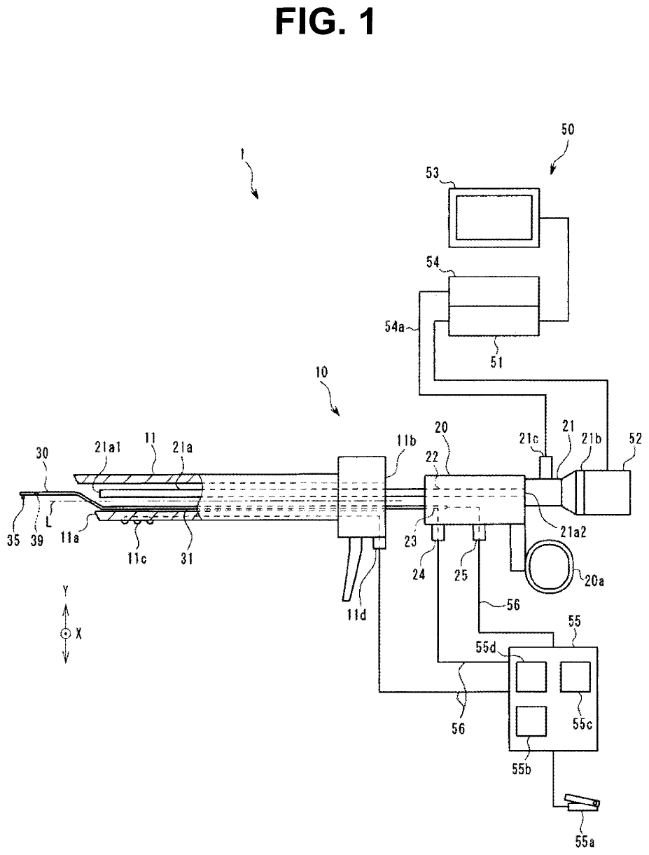

FIG. 1 is a diagram showing a schematic configuration of an endoscope system 1. The endoscope system 1 is a device configured to treat (for example, to resect or to coagulate) tissue inside a subject under observation with an endoscope.

The endoscope system 1 according to the present embodiment includes a resectoscope 10, which is an endoscope, an electrode unit 30, and an external device 50. In the present embodiment, the subject is a human body as an example. Although the endoscope in the present embodiment is, as an example, an endoscope typically referred to as resectoscope, the endoscope may also be a flexible endoscope.

The resectoscope 10 includes a sheath 11, a slider 20, and a telescope 21.

The sheath 11 includes a tubular area along a linear longitudinal axis L. The sheath 11 is an area inserted from the outside of the subject to the inside of the subject during use of the resectoscope 10. The sheath 11 has openings on both ends in a direction along the longitudinal axis L. During use of the resectoscope 10, the telescope 21 and the electrode unit 30 described later are inserted into the sheath 11.

Note that an outer sheath for introducing a perfusate into the subject is arranged on an outer periphery of the sheath 11. A configuration for introducing the perfusate into the subject, such as the outer sheath, is well-known and description of such a configuration is omitted. In the present embodiment, the perfusate is, for example, an electrolyte solution such as physiological saline and is electrically conductive.

Of both ends of the sheath 11 in the direction along the longitudinal axis L, an end on a side to be inserted into the subject is referred to as a “distal end 11a”, and an end on an opposite side to the distal end 11a is referred to as a “proximal end 11b”. The proximal end 11b of the sheath 11 is exposed to the outside of the subject during use of the resectoscope 10.

For the sake of description hereinafter, a first axis X and a second axis Y are defined, which are a pair of axes orthogonal to the longitudinal axis L and orthogonal to each other. In a direction along the first axis X, one side is defined as a right side and the other side is defined as a left side. In a direction along the second axis Y, one side is defined as an upper side and the other side is defined as a lower side. In the present embodiment, in an image picked up by using the telescope 21, a horizontal direction is substantially parallel to the first axis X, and a perpendicular direction is substantially parallel to the second axis Y, as an example. The upper side and the right side are an upper side and a right side in the image picked up by using the telescope 21.

A collection electrode 11c configured with an electrically conductive material is exposed at least to the surface in the vicinity of the distal end 11a of the sheath 11. Note that it may also be configured that the entire sheath 11 is configured with an electrically conductive material such as a metal and the entire surface of the sheath 11 functions as the collection electrode 11c.

A sheath connector 11d is provided in the vicinity of the proximal end 11b of the sheath 11. The sheath connector 11d is electrically connected to the collection electrode 11c. A cable 56 is connected to the sheath connector 11d. The cable 56 electrically connects the sheath connector 11d and a high-frequency power control device 55 of the external device 50.

The slider 20 is arranged on a side of the proximal end 11b of the sheath 11. The slider 20 moves relative to the sheath 11 in the direction along the longitudinal axis L. The slider 20 is provided with a handle 20a. When a user applies a force to the handle 20a with fingers, the slider 20 moves relative to the sheath 11 in the direction along the longitudinal axis L. Note that a mechanism for guiding the slider 20 in a relatively movable manner to the sheath 11 is similar to the mechanism of the conventional resectoscope 10, and therefore illustration and description of the mechanism are omitted.

The slider 20 includes a scope holding portion 22, an electrode unit holding portion 23, an electrode connector 24, and a detection connector 25. The scope holding portion 22 holds the telescope 21.

The telescope 21 is an area for optically observing the inside of the subject. The telescope 21 is provided with an elongated insertion portion 21a, an eye piece 21b, and a light source connection portion 21c. The insertion portion 21a is inserted into the sheath 11 in a state in which the telescope 21 is fixed onto the scope holding portion 22.

An observation window and an illumination light emission window are provided on a distal end portion 21a1 of the insertion portion 21a. The eye piece 21b and the light source connection portion 21c are provided on a proximal end portion 21a2 of the insertion portion 21a.

An image pickup unit 52 is attached to the eye piece 21b. The image pickup unit 52 is electrically connected to a video processor 51 of the external device 50. An image display device 53 is electrically connected to the video processor 51. One end of an optical fiber cable 54a is connected to the light source connection portion 21c. The other end of the optical fiber cable 54a is connected to a light source device 54 of the external device 50.

An image of a field of view from the observation window provided on the distal end portion 21a1 of the insertion portion 21a is picked up by the image pickup unit 52 and displayed on the image display device 53. Illumination light emitted from the light source device 54 is emitted from the illumination light emission window provided on the distal end portion 21a1 of the insertion portion 21a. Configurations of the telescope 21 and the external device 50 connected to the telescope 21 are similar to the configurations in the conventional resectoscope 10, and therefore detailed description of the configurations is omitted.

The electrode unit holding portion 23 holds the electrode unit 30 described later. The electrode connector 24 and the detection connector 25 are electrically connected to the electrode unit 30 held by the electrode unit holding portion 23. A cable 56 is connected to the electrode connector 24 and the detection connector 25. The cable 56 electrically connects the electrode connector 24 and the detection connector 25, respectively, with the high-frequency power control device 55 of the external device 50.

The electrode unit 30 includes an area inserted into the sheath 11 in a state of being fixed onto the electrode unit holding portion 23. The slider 20 moves together with the telescope 21 and the electrode unit 30, relative to the sheath 11 in the direction along the longitudinal axis L. A portion of the electrode unit 30 may protrude from the distal end 11a of the sheath 11. As described later, a treatment electrode 35 is provided in the area of the electrode unit 30 protruding from the distal end 11a of the sheath 11.

The electrode unit 30, the collection electrode 11c, and the high-frequency power control device 55 configure a so-called bipolar electro-surgical apparatus. The high-frequency power control device 55 includes a switch 55a, a resistance detection unit 55b as the resistance detection circuit, a processor 55c, and an information output unit 55d.

The switch 55a is, for example, a foot switch operated by a user's foot. The switch 55a is a device for the user to input an instruction of outputting the high-frequency current to the high-frequency power control device 55.

The high-frequency power control device 55 switches between presence and absence of output of the high-frequency current to the electrode unit 30 on the basis of an operation on the switch 55a by the user and a determination process described later. The high-frequency current outputted from the high-frequency power control device 55 flows among the treatment electrode 35, the perfusate, and the collection electrode 11c inside the subject. In a state in which the high-frequency power control device 55 outputs the high-frequency current, tissue in the subject in contact with the treatment electrode 35 generates heat, whereby the tissue is treated (for example, resected or coagulated).

The resistance detection unit 55b detects a resistance value of a current flowing in the electrode unit 30. The processor 55c includes hardware configured to control the operation of the high-frequency power control device 55. The processor 55c operates depending on a program stored in a storage device not illustrated. The information output unit 55d outputs information directed to the user. The information output unit 55d includes, for example, a display device configured to display images and characters, a light emission device configured to emit light, a speaker configured to produce sound, a vibrator configured to produce vibration, or a combination of the aforementioned devices. Alternatively, the information output unit 55d may be in a mode of outputting information directed to the user through the image display device 53. Operations of the resistance detection unit 55b, the processor 55c, and the information output unit 55d are described later.

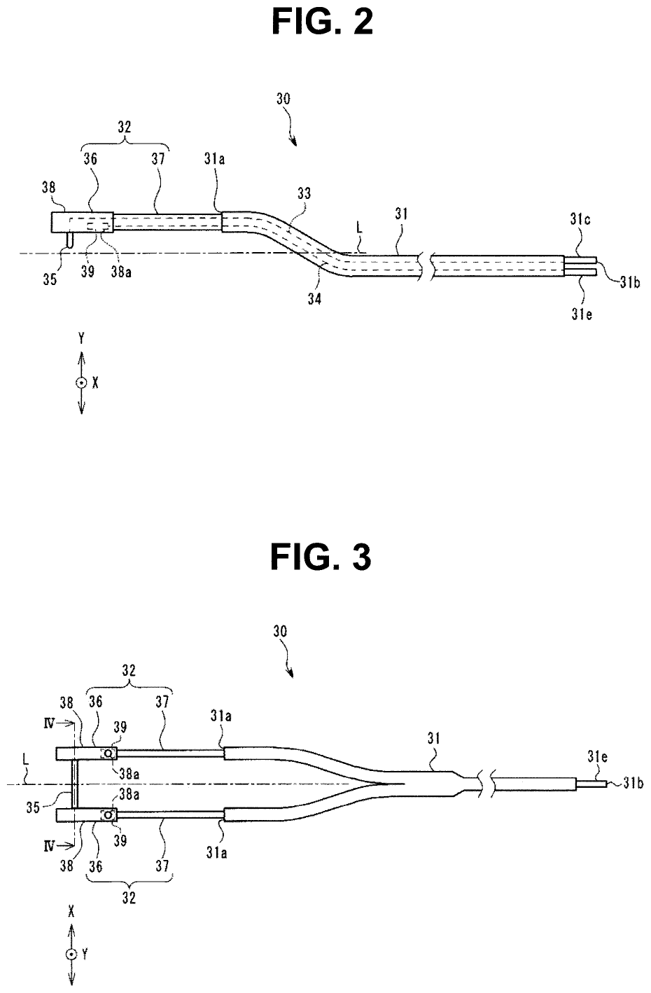

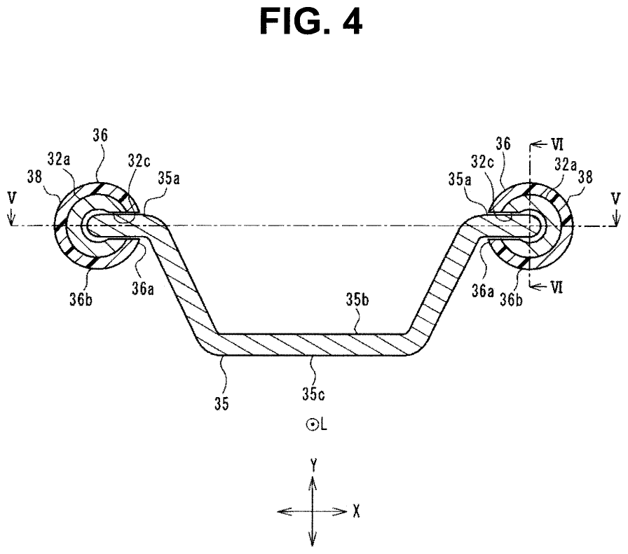

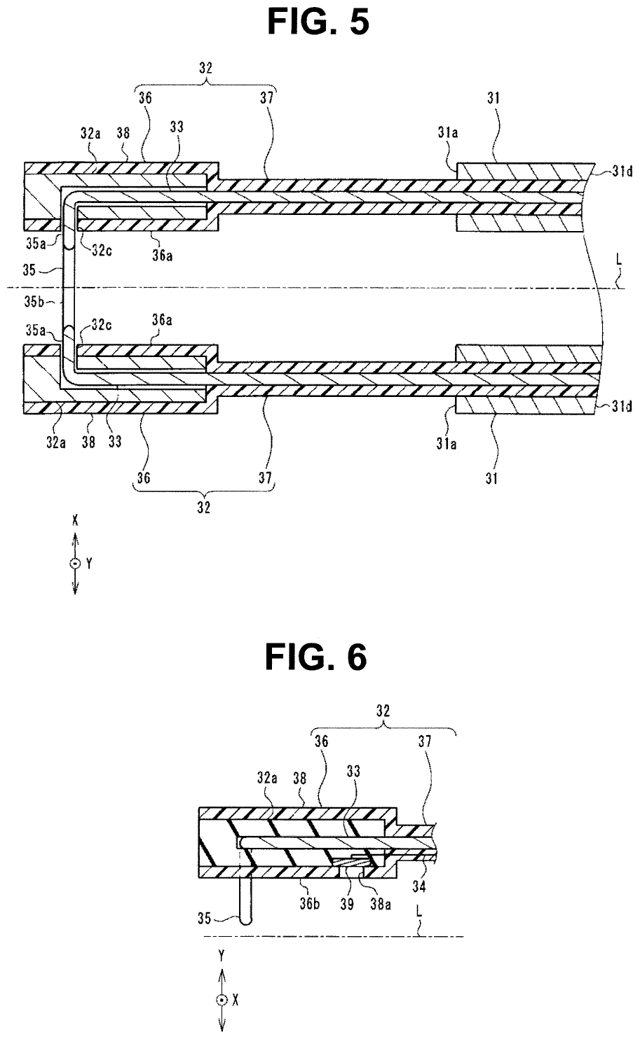

FIG. 2 is a diagram showing the electrode unit 30 seen from left along the first axis X. In FIG. 2, an upper side of the diagram is the upper side. FIG. 3 is a diagram showing the electrode unit 30 seen from below along the second axis Y. In FIG. 3, an upper side of the diagram is the left side. FIG. 4 is a cross-sectional view taken along a line IV-IV in FIG. 3. In FIG. 4, an upper side of the diagrams is the upper side, and a right side of the diagrams is the left side. FIG. 5 is a cross-sectional view taken along a V-V line in FIG. 4. In FIG. 5, an upper side of the diagram is the right side. FIG. 6 is a cross-sectional view taken along a line VI-VI in FIG. 4. In FIG. 4, an upper side of the diagram is the upper side.

As shown in FIG. 2 and FIG. 3, the electrode unit 30 has an elongated shape the longitudinal direction of which is a direction along the longitudinal axis L. The electrode unit 30 includes a proximal end rigid portion 31 as the proximal end rigid member, an electrode supporting portion 32 as the electrode supporting member, and the treatment electrode 35.

The proximal end rigid portion 31 is an area fixed to the electrode unit holding portion 23 of the resectoscope 10. The electrode supporting portion 32 described later is joined to a distal end 31a of the proximal end rigid portion 31. An electrical connection portion 31c and a detection connection portion 31e are provided on a proximal end 31b of the proximal end rigid portion 31.

The electrical connection portion 31c is electrically connected to the electrode connector 24 of the resectoscope 10 in a state in which the proximal end rigid portion 31 is fixed to the electrode unit holding portion 23. The electrical connection portion 31c is electrically connected to the treatment electrode 35 via an electrically conductive wire 33 inserted into the electrode unit 30.

The detection connection portion 31e is electrically connected to the detection connector 25 of the resectoscope 10 in a state in which the proximal end rigid portion 31 is fixed to the electrode unit holding portion 23. In addition, the detection connection portion 31e is electrically connected to the detection electrode 39 as the detection sensor described later, via an electrically conductive wire 34 (illustrated in FIG. 2) inserted into the electrode unit 30.

The electrode supporting portion 32 supports the treatment electrode 35 and the detection electrode 39. The electrode supporting portion 32 is an area protruding from the distal end 11a of the sheath 11 during use of the resectoscope 10. The electrode supporting portion 32 includes distal end rigid portions 36 as the one or two distal end rigid member(s), and one or two elastic region(s) 37. The treatment electrode 35 and the detection electrode 39 are fixed to the distal end rigid portion 36.

The elastic region 37 joins a proximal end of the distal end rigid portion 36 with a distal end of the proximal end rigid portion 31. The bending rigidity of the elastic region 37 is lower than the bending rigidity of the distal end rigid portion 36 and the proximal end rigid portion 31.

The treatment electrode 35 includes an electrically conductive linear member such as a metal wire. The treatment electrode 35 protrudes from a surface of the distal end rigid portion 36.

The treatment electrode 35 has a loop shape protruding from one point on the surface of the distal end rigid portion 36 to the outside of the distal end rigid portion 36, and entering from a different point to the inside of the distal end rigid portion 36. More specifically, the treatment electrode 35 includes, in two points spaced apart from each other on the surface of the distal end rigid portion 36, a pair of base portions 35a supported by the distal end rigid portion 36 and a spanning portion 35b connecting the pair of base portions 35a in a state of being spaced apart from the surface of the distal end rigid portion 36.

As shown in FIG. 4, the spanning portion 35b is substantially angled-U shaped or substantially U shaped when seen in a direction along the longitudinal axis L. When seen in the direction along the first axis X, an apex portion 35c of the spanning portion 35b protrudes from the base portion 35a in a direction intersecting the longitudinal axis L.

The pair of base portions 35a is electrically connected to the wire 33 inside the distal end rigid portion 36. As shown in FIG. 4 and FIG. 5, in the present embodiment, the wire 33 and the treatment electrode 35 are configured with the same metallic linear member, as an example.

The detection electrode 39 is arranged to be exposed to the outside on a lower end face 36b, which is a face directed in the protrusion direction of the treatment electrode 35, of the external surface of the distal end rigid portion 36. The detection electrode 39 is arranged on the proximal end side relative to the treatment electrode 35.

More specifically, the electrode supporting portion 32 of the present embodiment is provided with two distal end rigid portions 36. Each of the distal end rigid portions 36 has a columnar external shape the longitudinal direction of which is a direction along the longitudinal axis L. Note that a cross-section of the distal end rigid portion 36 in the present embodiment illustrated in the drawings is substantially round. However, a cross-section of the distal end rigid portion 36 may also be either a parallelogram shape or other polygonal shapes.

The two distal end rigid portions 36 are arranged in substantially the same position in the direction along the longitudinal axis L, and arranged to be spaced apart from each other in the direction along the first axis X (lateral direction). In other words, the two distal end rigid portions 36 are arranged such that an overlapping part exists when seen in the direction along the first axis X. Therefore, the two distal end rigid portions 36 respectively have opposed faces 36a facing each other in the direction along the first axis X.

Note that the expression “faces facing each other” as used herein refers to a surface oriented substantially to the left side of the distal end rigid portion 36 arranged on the right side, and a surface oriented substantially to the right side of the distal end rigid portion 36 arranged on the left side. In other words, the opposed faces 36a are parts facing a space between the two distal end rigid portions 36. Therefore, the opposed faces 36a of the two distal end rigid portions 36 are not required to have respective parts parallel to each other.

The pair of base portions 35a of the treatment electrode 35 are arranged respectively on the two distal end rigid portions 36. In other words, the treatment electrode 35 is the metal wire 33 spanning the two distal end rigid portions 36.

The pair of base portions 35a are arranged to protrude along the first axis X, respectively from the opposed faces 36a of the two distal end rigid portions 36. The pair of base portions 35a are arranged in substantially the same position in the direction along the longitudinal axis L. In other words, the pair of base portions 35a protrude respectively from the pair of opposed faces 36 along the first axis X, to get closer to each other.

The spanning portion 35b connects distal end portions of the pair of base portions 35a. The spanning portion 35b is curved in a downward convex shape from the pair of base portions 35a, when seen in the direction along the longitudinal axis L. As shown in FIG. 4, the apex portion 35c of the spanning portion 35b is positioned on the lower side of a lower end face 36b, facing the lower side, of the two distal end rigid portions 36.

The treatment electrode 35 having the configuration described in the foregoing is exposed to the outside only within a space between the two distal end rigid portions 36 when seen in the direction along the second axis Y. In other words, an area of the treatment electrode 35 exposed to the outside is arranged not to overlap the two distal end rigid portions 36 when seen in the direction along the second axis Y.

As shown in FIG. 4 and FIG. 5, each of the distal end rigid portions 36 is configured with a ceramic pipe 32a and a covering portion 38. The ceramic pipe 36c and the covering portion 38 are electrically insulating. The ceramic pipe 32a is a hollow member into which the wire 33 is inserted. The covering portion 38 is a tube made of a resin and covers the ceramic pipe 32a. On respective lateral faces of the ceramic pipe 32a and the covering portion 38, a through hole 32c that holds the base portion 35a of the treatment electrode 35 is formed.

The electrode supporting portion 32 of the present embodiment is provided with two elastic regions 37 as the elastic member, as an example. The two elastic regions 37 are connected respectively to the proximal ends of the two distal end rigid portions 36. Note that the electrode supporting portion 32 may also be configured to have one elastic region 37 connected to the proximal ends of both of the two distal end rigid portions 36.

The elastic region 37 of the present embodiment is configured with the covering portion 38, which is a tube made of an electrically insulating resin. In the present embodiment, the covering portion 38 of the distal end rigid portion 36 and the covering portion 38 of the elastic region 37 are the same member continuous in the direction along the longitudinal axis L, as an example. The wire 33 is inserted into the covering portion 38 of the elastic region 37. In other words, in the present embodiment, the ceramic pipe 32a inserted into the covering portion 38 plays a role in making the bending rigidity of the distal end rigid portions 36 higher than the bending rigidity of the elastic region 37.

The proximal end rigid portion 31 of the present embodiment is configured with the covering portion 38, which is a tube made of a resin, and a metal pipe 31d. In the present embodiment, the covering portion 38 of the proximal end rigid portion 31 and the covering portion 38 of the elastic region 37 are the same member continuous in the direction along the longitudinal axis L, as an example. The wire 33 is inserted into the covering portion 38 of the proximal end rigid portion 31. The metal pipe 31d covers the outer periphery of the covering portion 38. In other words, in the present embodiment, the metal pipe 31d plays a role in making the bending rigidity of the proximal end rigid portions 31 higher than the bending rigidity of the elastic region 37.

As shown in FIG. 3 and FIG. 6, the detection electrode 39 is arranged on the proximal end side relative to the treatment electrode 35 on the respective lower end faces 36b of the two distal end rigid portions 36. In other words, the electrode unit 30 of the present embodiment is provided with a plurality of detection electrodes 39 arranged to be spaced apart from each other. In the present embodiment, the detection electrode 39 is provided inside the covering portion 38, and is exposed to the outside through a through hole 38a penetrating the covering portion 38, as an example.

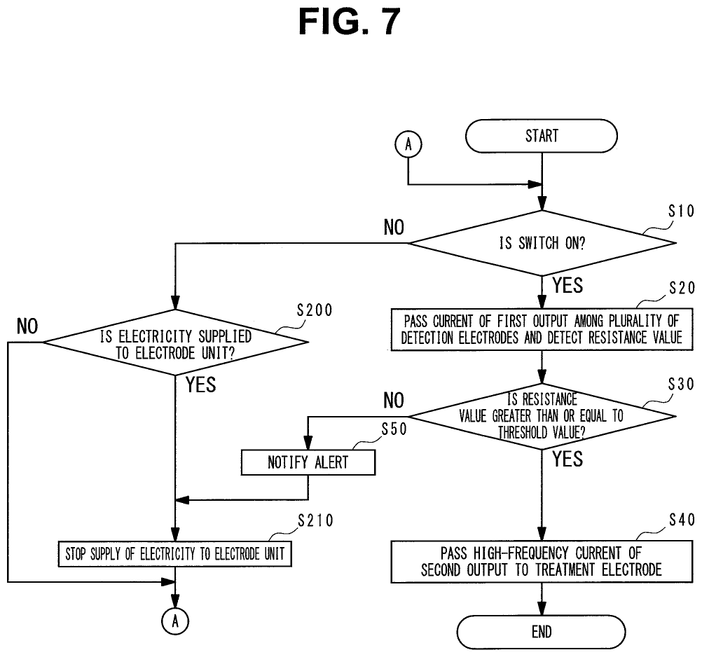

Next, the operation of the high-frequency power control device 55 of the present embodiment is described with reference to the flowchart of FIG. 7. The process of the flowchart of FIG. 7 is executed repeatedly by the processor 55c in a predetermined cycle. At the moment of starting the process of the flowchart of FIG. 7, the high-frequency power control device 55 is in a state in which output of a current is stopped.

First, in step S10, the processor 55c determines whether or not the switch 55a is in an ON state. The switch 55a is in the ON state when the instruction of outputting the high-frequency current is inputted by the user.

When it has been determined that the switch 55a is not in the ON state in the determination in step S10, the processor 55c proceeds to step S200. In step S200, the processor 55c determines whether or not the high-frequency power control device 55 is supplying electricity to the electrode unit 30.

When it has been determined that electricity is supplied to the electrode unit 30 in the determination in step S200, the processor 55c proceeds to step S210. In step S210, the processor 55c stops supply of electricity from the high-frequency power control device 55 to the electrode unit 30 and returns to step S10. When it has been determined that electricity is not supplied to the electrode unit 30 in the determination in step S200, the processor 55c returns to step S10. In other words, when the switch 55a is not in the ON state, the high-frequency power control device 55 does not supply electricity to the electrode unit 30.

On the other hand, when it has been determined that the switch 55a is in the ON state, the processor 55c executes processes of step S20 and later.

In step S20, the processor 55c detects an electrical resistance value among the plurality of detection electrodes 39. More specifically, the processor 55c passes a minute current of a first output among the plurality of detection electrodes 39 and detects the resistance value of the current by means of the resistance detection unit 55b. The first output is smaller than a second output, which is an output of the high-frequency current to be passed to the treatment electrode 35 upon treatment (for example, resection or coagulation) of the tissue.

Next, in step S30, the processor 55c determines whether or not the resistance value detected by the resistance detection unit 55b is greater than or equal to a predetermined threshold value Th. The threshold value Th of the treatment is a resistance value slightly greater than a resistance value in the case in which the plurality of detection electrodes 39 are exposed into an electrically conductive perfusate. In the present embodiment, the electrically conductive perfusate is physiological saline.

When it has been determined that the resistance value is greater than or equal to the threshold value Th in the determination in step S30, the processor 55c proceeds to step S40. In step S40, the processor 55c starts outputting the high-frequency current of the second output to the treatment electrode 35. As described above, the high-frequency current of the second output is to be outputted upon treatment (for example, resection or coagulation) of the tissue. In other words, when the electrical resistance value among the plurality of detection electrodes 39 is greater than or equal to the predetermined threshold value, the processor 55c permits output of the high-frequency current of the second output to the treatment electrode 35.

When it has been determined that the resistance value is less than the threshold value Th in the determination in step S30, the processor 55c proceeds to step S50. In step S50, the processor 55c outputs information indicating an alert by means of the information output unit 55b. The information indicating an alert includes information notifying the user that the attitude of the electrode unit 30 is not appropriate. The method for outputting the information from the information output unit 55b may be either generation of sound or output of an image to the image display device 53. And then the processor 55c proceeds to step S210 and stops supply of electricity from the high-frequency power control device 55 to the electrode unit 30. In other words, when the electrical resistance value among the plurality of detection electrodes 39 is less than the predetermined threshold value, the processor 55c inhibits output of the high-frequency current of the second output to the treatment electrode 35.

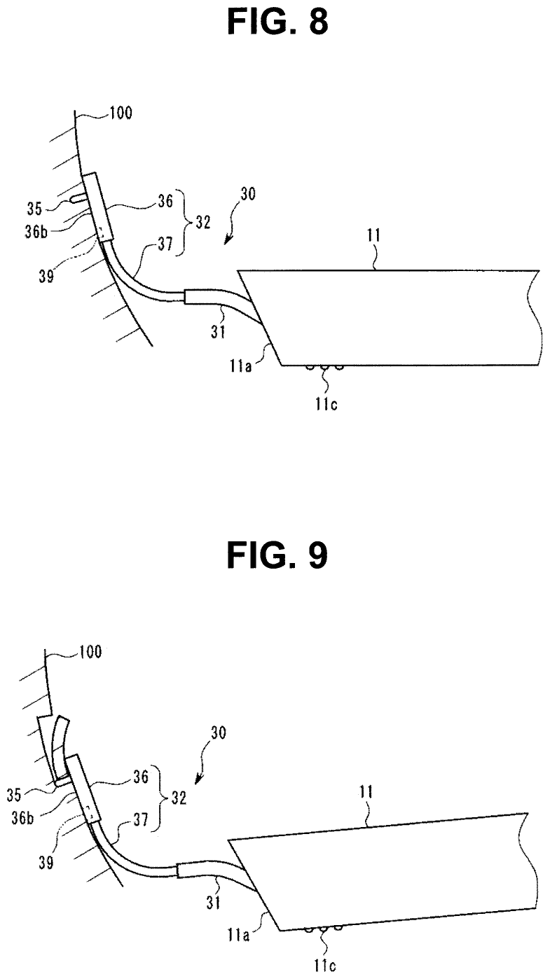

FIG. 8 and FIG. 9 show schematic views of a manner of resecting tissue in an organ 100 in the subject by using the electrode unit 30 and the endoscope system 1 of the present embodiment.

In the case of resecting tissue inside the organ 100 by using the electrode unit 30, a user first orients the electrode supporting portion 32 such that the lower end face 36b of the distal end rigid portion 36 faces the tissue inside the organ 100. And then the user brings the electrode supporting portion 32 into contact with a wall surface of the organ 100 such that the lower end face 36b of the distal end rigid portion 36 is in contact with the tissue, as shown in FIG. 8. During this operation, the treatment electrode 35 is substantially buried in the tissue while the tissue is not dissected. In addition, the organ 100 is filled with the electrically conductive perfusate. Note that the method of inserting the electrode unit 100 and the sheath 11 of the resectoscope 10 into the organ 100 as well as the method of filling the organ 100 with the perfusate are the same as the method in the case of the conventional electrode unit, and therefore description of the methods is omitted.

Next, the user operates the switch 55a. During this operation, as shown in FIG. 8, when the lower end face 36b of the distal end rigid portion 36 is entirely in contact with the tissue and the wall surface of the tissue is substantially in parallel to the lower end face 36b, the plurality of detection electrodes 39 are covered by the tissue. In this case, the tissue is interposed in the electrical connection among the plurality of detection electrodes 39, whereby the electrical resistance value among the plurality of detection electrodes 39 is greater than or equal to the predetermined threshold value Th.

Therefore, as shown in FIG. 8, when the lower end face 36b of the distal end rigid portion 36 is entirely in contact with the tissue, the high-frequency power control device 55 starts outputting the high-frequency current of second electric power in response to the operation on the switch 55a by the user. As a result, the high-frequency current flows from the treatment electrode 35 to the collection electrode 11c through the perfusate, whereby the tissue in contact with the treatment electrode 35 generates heat and is resected.

As described above, the treatment electrode 35 is arranged not to overlap the distal end rigid portions 36 when seen in the direction along the second axis Y (from the lower side). Therefore, the depth to which the treatment electrode 35 can enter the tissue is limited in the state in which the lower end face 36b of the distal end rigid portion 36 is in contact with the tissue. In other words, the lower end face 36b of the distal end rigid portion 36 functions as a stopper configured to restrict the depth to which the electrode 35 enters the tissue.

If, unlike the present embodiment, the electrode 35 was arranged to overlap the lower end face 36b of the distal end rigid portion 36 when seen from the lower side, the lower end face 36b would be pressed against the tissue cut off by the electrode 35. In this case, the force of the lower end face 36b restricting the proceeding of the electrode 35 into the tissue may be weaker than in the present embodiment. The present embodiment can avoid such a state, and enables reliable restriction of the depth to which the electrode 35 enters the tissue.

Therefore, in the present embodiment, even when the force with which the user presses the electrode supporting portion 32 against the wall surface of the organ 100 varies, the treatment electrode 35 can be prevented from further entering the tissue, from the state in which the distal end rigid portion 36 is in contact with the tissue.

And then as shown in FIG. 9, the user moves the resectoscope 10 to move the electrode supporting portion 32 along the wall surface of the organ 100. As a result, the treatment electrode 35 moves in a direction along the wall surface inside the tissue, whereby a tissue piece of a predetermined thickness is resected.

As described above, even when the force with which the user presses the electrode supporting portion 32 against the wall surface of the organ 100 varies, the depth to which the treatment electrode 35 enters the tissue is kept constant. Also, even when the force with which the user presses the resectoscope 10 in a tissue direction varies, the elastic portion 37 bends, whereby the change in the force of pressing the electrode 35 in the tissue direction is kept substantially constant. As a result, an amount of the tissue to be deformed by the distal end rigid portions 36 is also kept substantially constant, whereby the depth to which the electrode 35 enters the tissue is also kept substantially constant. In the present embodiment, even when movement of the resectoscope 10 by the user does not follow the shape of the wall surface of the organ 100 and a distance between the wall surface of the organ 100 and the distal end 11a of the sheath 11 varies, the elastic region 37 elastically deforms to keep the state in which the distal end rigid portions 36 is in contact with the tissue. As described above, when the distal end rigid portions 36 is in contact with the tissue, the depth to which the treatment electrode 35 enters the tissue is kept constant.

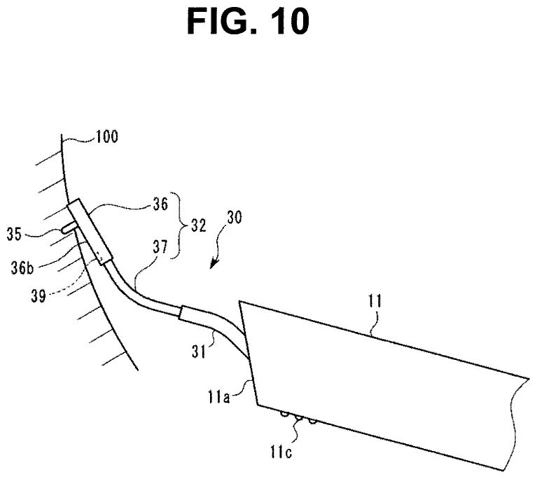

In addition, as shown in FIG. 10, when the lower end face 36b of the distal end rigid portion 36 is not entirely in contact with the tissue, the plurality of detection electrodes 39 are exposed into the perfusate. In this case, since the electrical resistance value among the plurality of detection electrodes 39 is less than the predetermined threshold value Th, even when the user operates on the switch 55a, the high-frequency power control device 55 does not output the high-frequency current of the second electric power.

As shown in FIG. 10, when the lower end face 36b of the distal end rigid portion 36 is not entirely in contact with the tissue and the wall surface of the tissue is not substantially in parallel to the lower end face 36b, the attitude of the treatment electrode 35 with respect to the tissue is different from the desired state, and the depth to which the treatment electrode 35 enters the tissue may be different from the predetermined depth.

In a case in which the attitude of the treatment electrode 35 with respect to the tissue is different from the desired state, the electrode unit 30 and the endoscope system 1 according to the present embodiment stop outputting the high-frequency current from the high-frequency power control device 55. In other words, by using the electrode unit 30 and the endoscope system 1 according to the present embodiment, the depth to which the treatment electrode 35 enters the tissue can be maintained constant during a period of carrying out resection of the tissue by passing the high-frequency current from the treatment electrode 35.

As explained in the foregoing, the electrode unit 30 and the endoscope system 1 of the present embodiment can keep constant the depth to which the treatment electrode 35 enters the tissue, even when a trajectory of the treatment electrode 35 moved by the user is unstable, or when the force applied by the user to the treatment electrode 35 varies. Therefore, the electrode unit 30 and the endoscope system 1 of the present embodiment facilitate control of the thickness of the resected tissue.

Note that, although in the electrode unit 30 and the endoscope system 1 according to the present embodiment, it is detected whether or not the lower end face 36b of the distal end rigid portion 36 is entirely in contact with the tissue on the basis of the electric resistance among the plurality of detection electrodes 39, the method for the detection is not limited to the present embodiment and may also be in a mode of using a sensor provided in the electrode unit 30.

For example, the electrode unit 30 may be in a mode in which a pressure sensor configured to detect pressure applied to the lower end face 36b is provided. In this case, the processor 55c determines that the lower end face 36b of the distal end rigid portion 36 is entirely in contact with the tissue when the pressure detected by the pressure sensor is greater than or equal to a predetermined threshold value.

Alternatively, for example, the electrode unit 30 may be in a mode in which a ranging sensor configured to measure a distance between the lower end face 36b and the tissue by means of acoustic waves or the like is provided. In this case, the processor 55c determines that the lower end face 36b of the distal end rigid portion 36 is entirely in contact with the tissue when the distance detected by the ranging sensor is less than or equal to a predetermined threshold value.

### Second Embodiment

Hereinafter, the second embodiment of the present invention is described. In the following description, only differences from the first embodiment are explained, and constitutive elements similar to the constitutive elements of the first embodiment are denoted by the same reference symbols and description of these constitutive elements is omitted as appropriate.

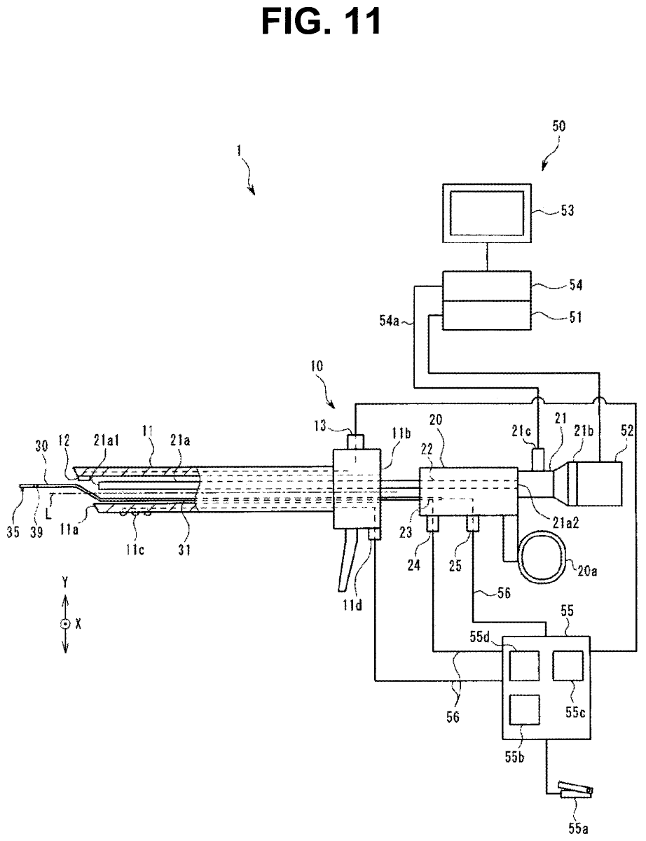

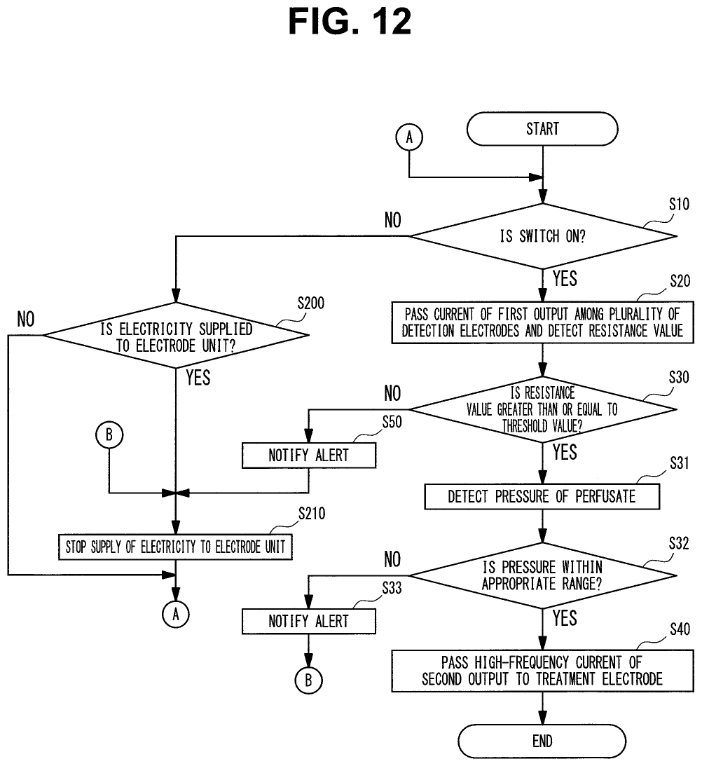

The endoscope system 1 according to the present embodiment shown in FIG. 11 is provided with a pressure sensor 12. The pressure sensor 12 detects pressure of the perfusate during use of the endoscope system 1, and outputs a detection result to the processor 55c.

A site where the pressure sensor 12 is arranged is not particularly limited, and in the present embodiment, the pressure sensor 12 is arranged in a distal end portion of the sheath 11, as an example. A sensor connector 13 is provided in the vicinity of the proximal end 11b of the sheath 11. A cable 56 is connected to the sensor connector 13. The cable 56 electrically connects the sensor connector 13 and the high-frequency power control device 55.

Next, the operation of the high-frequency power control device 55 of the present embodiment is described. FIG. 12 is a flowchart showing an operation of the high-frequency power control device 55 of the present embodiment. The flowchart of FIG. 12 includes step S31, step S32, and step S33 in addition to the flowchart of the first embodiment (FIG. 7).

The process of the flowchart of FIG. 12 is executed repeatedly by the processor 55c in a predetermined cycle. At the moment of starting the process of the flowchart of FIG. 12, the high-frequency power control device 55 is in a state in which output of a current is stopped.

First, in step S10, the processor 55c determines whether or not the switch 55a is in an ON state. The switch 55a is in the ON state when the user inputs the instruction of outputting the high-frequency current.

When it has been determined that the switch 55a is not in the ON state in the determination in step S10, the processor 55c proceeds to step S200. In step S200, the processor 55c determines whether or not the high-frequency power control device 55 is supplying electricity to the electrode unit 30.

When it has been determined that electricity is supplied to the electrode unit 30 in the determination in step S200, the processor 55c proceeds to step S210. In step S210, the processor 55c stops supply of electricity from the high-frequency power control device 55 to the electrode unit 30 and returns to step S10. When it has been determined that electricity is not supplied to the electrode unit 30 in the determination in step S200, the processor 55c returns to step S10. In other words, when the switch 55a is not in the ON state, the high-frequency power control device 55 does not supply electricity to the electrode unit 30.

On the other hand, when it has been determined that the switch 55a is in the ON state, the processor 55c executes processes of step S20 and later.

In step S20, the processor 55c detects an electrical resistance value among the plurality of detection electrodes 39. More specifically, the processor 55c passes a minute current of a first output among the plurality of detection electrodes 39 by means of the resistance detection unit 55b and detects the resistance value of the current. The first output is smaller than a second output, which is an output of the high-frequency current to be passed to the treatment electrode 35 upon treatment (for example, resection or coagulation) of the tissue.

Next, in step S30, the processor 55c determines whether or not the resistance value detected by the resistance detection unit 55b is greater than or equal to a predetermined threshold value Th. Here, the threshold value Th of the treatment is a resistance value slightly greater than a resistance value of the case in which the plurality of detection electrodes 39 are exposed into an electrically conductive perfusate. In the present embodiment, the electrically conductive perfusate is physiological saline.

When it has been determined that the resistance value is greater than or equal to the threshold Th in the determination in step S30, the processor 55c proceeds to step S31. In step S31, the processor 55c detects pressure of the perfusate inside the organ 100 by means of the pressure sensor 12.

Next, in step S32, the processor 55c determines whether or not the pressure of the perfusate inside the organ 100 is within a predetermined appropriate range.

When it has been determined that the pressure of the perfusate inside the organ 100 is within the predetermined appropriate range in the determination in step S32, the processor 55c proceeds to step S40. In step S40, the processor 55c starts outputting the high-frequency current of the second output to the treatment electrode 35. As described above, the high-frequency current of the second output is to be outputted upon treatment (for example, resection or coagulation) of the tissue.

In other words, when the electrical resistance value among the plurality of detection electrodes 39 is greater than or equal to the predetermined threshold value, and the pressure of the perfusate inside the organ 100 is within the predetermined range, the processor 55c permits output of the high-frequency current of the second output to the treatment electrode 35.

Getting back to the description of the branch at step S30. When it has been determined that the resistance value is less than the threshold value Th in step S30, the processor 55c proceeds to step S50. In step S50, the processor 55c outputs information indicating an alert by means of the information output unit 55b. Here, the information indicating an alert includes information notifying the user that the attitude of the electrode unit 30 is not appropriate. And then the processor 55c proceeds to step S210 and stops supply of electricity from the high-frequency power control device 55 to the electrode unit 30. In other words, when the electrical resistance value among the plurality of detection electrodes 39 is less than the predetermined threshold value, the processor 55c inhibits output of the high-frequency current of the second output to the treatment electrode 35.

Getting back to the description of the branch at step S32. When it has been determined that the pressure of the perfusate inside the organ 100 is out of the predetermined appropriate range in the determination in step S32, the processor 55c proceeds to step S33. In step S33, the processor 55c outputs information indicating an alert by means of the information output unit 55b. The information indicating an alert includes information notifying the user that the pressure of the perfusate inside the organ 100 is not appropriate. And then the processor 55c proceeds to step S210 and stops supply of electricity from the high-frequency power control device 55 to the electrode unit 30.

In other words, when the pressure of the perfusate inside the organ 100 is out of the appropriate range, the processor 55c inhibits output of the high-frequency current of the second output to the treatment electrode 35, regardless of magnitude of the electrical resistance value among the plurality of detection electrodes 39.

When the pressure of the perfusate filling the organ 100 changes, the thickness of the wall surface of the organ 100 changes. For example, as the pressure of the perfusate filling the organ 100 increases, the organ 100 swells, whereby the wall surface of the organ 100 is thinned. On the other hand, as the pressure of the perfusate filling the organ 100 decreases, the organ 100 contracts, whereby the wall surface of the organ 100 is thickened.

Even when the depth to which the treatment electrode 35 enters the tissue is constant, the change in the thickness of the wall surface of the organ 100 may change the thickness of the tissue to be resected by the treatment electrode 35. As described above, the endoscope system 1 according to the present embodiment is enabled to pass the high-frequency current from the treatment electrode 35 to resect the tissue only when the pressure of the perfusate filling the organ 100 is within the predetermined appropriate range. In other words, the endoscope system 1 according to the present embodiment can maintain the thickness of the tissue to be resected substantially constant, due to the configuration of resecting the tissue only when the thickness of the wall surface of the organ 100 is within a predetermined range.

### Third Embodiment

Hereinafter, the third embodiment of the present invention is described. In the following description, only differences from the first embodiment are explained, and constitutive elements similar to the constitutive elements of the first embodiment are denoted by the same reference symbols and description of these constitutive elements is omitted as appropriate.

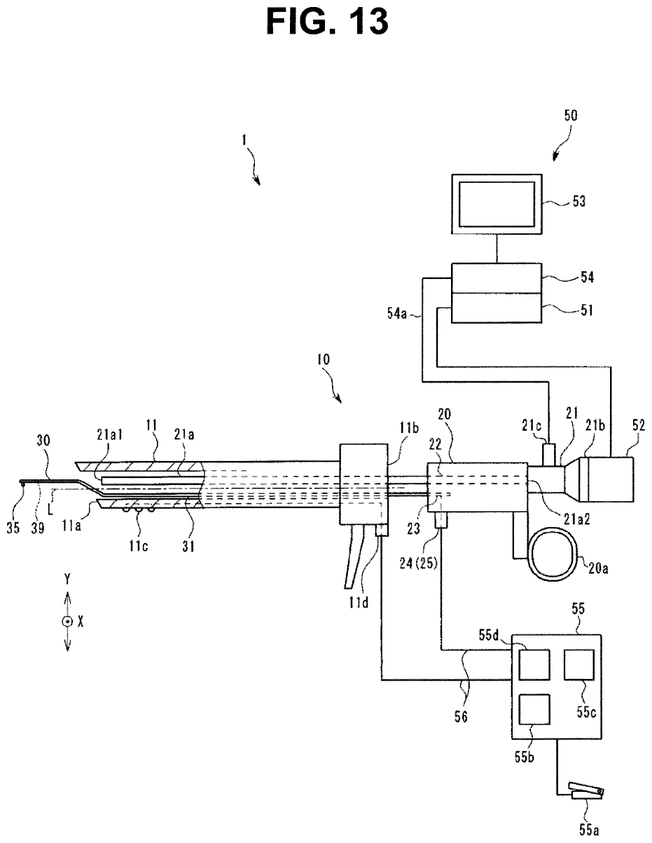

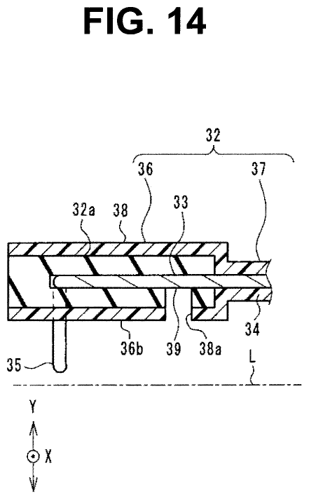

The endoscope system 1 according to the present embodiment shown in FIG. 13 and FIG. 14 is different from the first embodiment in the configurations of the detection electrode 39 and the resistance detection unit 55b.

As shown in FIG. 13, the detection electrode 39 of the present embodiment is electrically connected with the wire 33 and the treatment electrode 35. In the present embodiment, the detection electrode 39 is an area of the wire 33 exposed to the outside through the through hole 38a, as an example. The through hole 38a penetrates the covering portion 38 and the ceramic pipe 32a.

In the present embodiment, the electrical connection portion 31c of the electrode unit 30 acts also as the detection connection portion 31e. In addition, the electrode connector 24 of the resectoscope 10 acts also as the detection connector 25.

The resistance detection unit 55b detects an electrical resistance value between the detection electrode 39 and the collection electrode 11c. In other words, the resistance detection unit 55b passes a minute current of the first output between the wire 33 and the collection electrode 11c, and detects the resistance value of the current.

In the present embodiment as well, as shown in FIG. 8, when the lower end face 36b of the distal end rigid portion 36 is entirely in contact with the tissue and the wall surface of the tissue is substantially in parallel to the lower end face 36b, the through hole 38a is covered by the tissue as in the first embodiment. Therefore, as shown in FIG. 8, when the lower end face 36b of the distal end rigid portion 36 is entirely in contact with the tissue, the electrical resistance value between the detection electrode 39 and the collection electrode 11c is greater than or equal to the predetermined threshold value Th.

On the other hand, as shown in FIG. 10, when the lower end face 36b of the distal end rigid portion 36 is not entirely in contact with the tissue, the detection electrode 39 is exposed into the perfusate. Therefore, as shown in FIG. 10, when the lower end face 36b of the distal end rigid portion 36 is not entirely in contact with the tissue, the electrical resistance value between the detection electrode 39 and the collection electrode 11c is less than the predetermined threshold value Th.

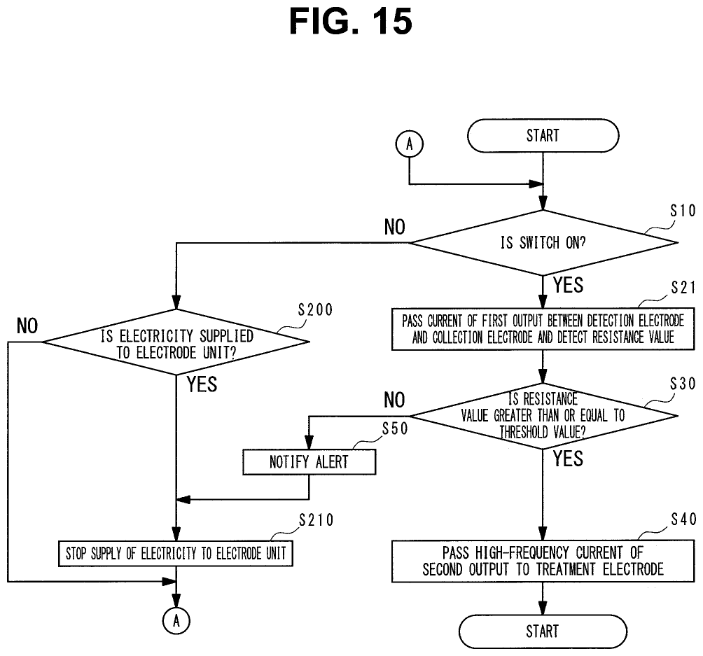

Next, the operation of the high-frequency power control device 55 of the present embodiment is described. FIG. 15 is a flowchart showing an operation of the high-frequency power control device 55 of the present embodiment. The flowchart of FIG. 15 is different from the flowchart of the first embodiment (FIG. 7) only in the process of step S21.

The process of the flowchart of FIG. 15 is executed repeatedly by the processor 55c in a predetermined cycle. At the moment of starting the process of the flowchart of FIG. 15, the high-frequency power control device 55 is in a state in which output of a current is stopped.

First, in step S10, the processor 55c determines whether or not the switch 55a is in an ON state. The switch 55a is in the ON state when the user inputs the instruction of outputting the high-frequency current.

When it has been determined that the switch 55a is not in the ON state in the determination in step S10, the processor 55c proceeds to step S200. In step S200, the processor 55c determines whether or not the high-frequency power control device 55 is supplying electricity to the electrode unit 30.

When it has been determined that electricity is supplied to the electrode unit 30 in the determination in step S200, the processor 55c proceeds to step S210. In step S210, the processor 55c stops supply of electricity from the high-frequency power control device 55 to the electrode unit 30 and returns to step S10. When it has been determined that electricity is not supplied to the electrode unit 30 in the determination in step S200, the processor 55c returns to step S10. In other words, when the switch 55a is not in the ON state, the high-frequency power control device 55 does not supply electricity to the electrode unit 30.

On the other hand, when it has been determined that the switch 55a is in the ON state, the processor 55c executes processes of step S21 and later.

In step S21, the processor 55c detects an electrical resistance value between the detection electrode 39 and the collection electrode 11c. More specifically, the processor 55c passes a minute current of a first output between the detection electrode 39 and the collection electrode 11c by means of the resistance detection unit 55b and detects the resistance value of the current. The first output is smaller than a second output, which is an output of the high-frequency current to be passed to the treatment electrode 35 upon treatment (for example, resection or coagulation) of the tissue.

Next, in step S30, the processor 55c determines whether or not the resistance value detected by the resistance detection unit 55b is greater than or equal to a predetermined threshold value Th. Here, the threshold value Th of the treatment is a resistance value slightly greater than a resistance value of the case in which the plurality of detection electrodes 39 are exposed into an electrically conductive perfusate. In the present embodiment, the electrically conductive perfusate is physiological saline.

When it has been determined that the resistance value is greater than or equal to the threshold Th in the determination in step S30, the processor 55c proceeds to step S40. In step S40, the processor 55c starts outputting the high-frequency current of the second output to the treatment electrode 35. As described above, the high-frequency current of the second output is to be outputted upon treatment (for example, resection or coagulation) of the tissue. In other words, when the electrical resistance value among the plurality of detection electrodes 39 is greater than or equal to the predetermined threshold value, the processor 55c permits output of the high-frequency current of the second output to the treatment electrode 35.

When it has been determined that the resistance value is less than the threshold value Th in the determination in step S30, the processor 55c proceeds to step S50. In step S50, the processor 55c outputs information indicating an alert, by means of the information output unit 55b. The information indicating an alert includes information notifying the user that the attitude of the electrode unit 30 is not appropriate. And then the processor 55c proceeds to step S210 and stops supply of electricity from the high-frequency power control device 55 to the electrode unit 30. In other words, when the electrical resistance value among the plurality of detection electrodes 39 is less than the predetermined threshold value, the processor 55c inhibits output of the high-frequency current of the second output to the treatment electrode 35.

As in the first embodiment, in a case in which the attitude of the treatment electrode 35 with respect to the tissue is different from the desired state, the electrode unit 30 and the endoscope system 1 according to the present embodiment stop outputting the high-frequency current from the high-frequency power control device 55. In other words, by using the electrode unit 30 and the endoscope system 1 according to the present embodiment, the depth to which the treatment electrode 35 enters the tissue can be maintained constant during a period of carrying out resection of the tissue by passing the high-frequency current from the treatment electrode 35.

As explained in the foregoing, the electrode unit 30 and the endoscope system 1 of the present embodiment can keep constant the depth to which the treatment electrode 35 enters the tissue, even when a trajectory of the treatment electrode 35 moved by the user is unstable, or when the force applied by the user to the treatment electrode 35 varies. Therefore, the electrode unit 30 and the endoscope system 1 of the present embodiment facilitate control of the thickness of the resected tissue.

### Fourth Embodiment

Hereinafter, the fourth embodiment of the present invention is described. In the following description, only differences from the first embodiment are explained, and constitutive elements similar to the constitutive elements of the third embodiment are denoted by the same reference symbols and description of these constitutive elements is omitted as appropriate.

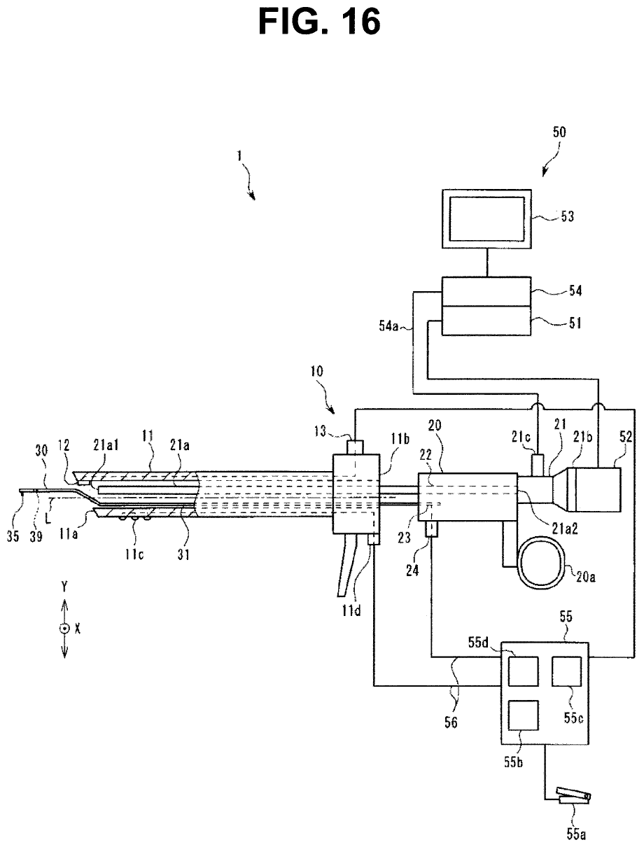

The endoscope system 1 according to the present embodiment shown in FIG. 16 is provided with a pressure sensor 12. The pressure sensor 12 detects pressure of the perfusate during use of the endoscope system 1, and outputs a detection result to the processor 55c.

A site where the pressure sensor 12 is arranged is not particularly limited, and in the present embodiment, the pressure sensor 12 is arranged in a distal end portion of the sheath 11, as an example. A sensor connector 13 is provided in the vicinity of the proximal end 11b of the sheath 11. A cable 56 is connected to the sensor connector 13. The cable 56 electrically connects the sensor connector 13 and the high-frequency power control device 55.

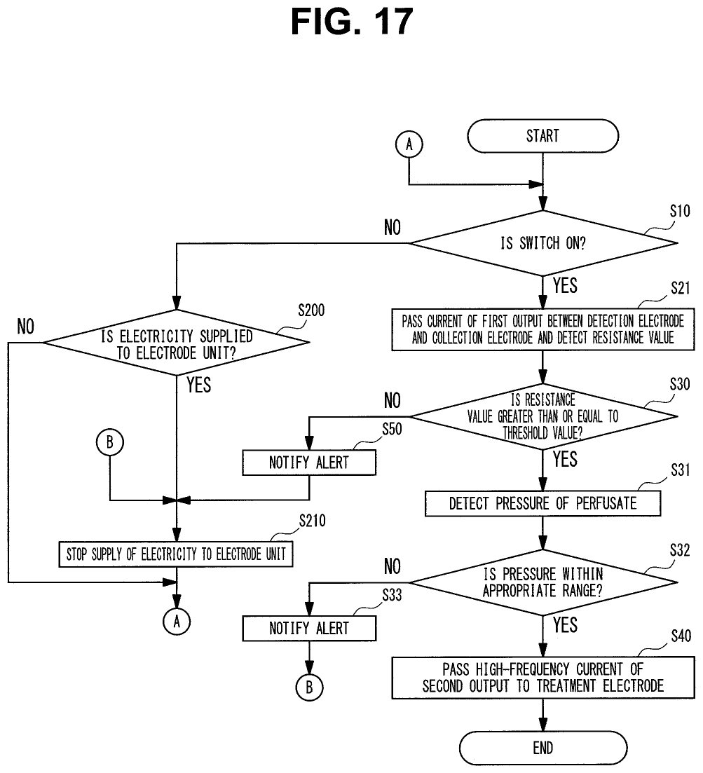

Next, the operation of the high-frequency power control device 55 of the present embodiment is described. FIG. 17 is a flowchart showing an operation of the high-frequency power control device 55 of the present embodiment. The flowchart of FIG. 17 includes step S31, step S32, and step S33 being added to the flowchart of the third embodiment (FIG. 15).

The process of the flowchart of FIG. 15 is executed repeatedly by the processor 55c in a predetermined cycle. At the moment of starting the process of the flowchart of FIG. 12, the high-frequency power control device 55 is in a state in which output of a current is stopped.

First, in step S10, the processor 55c determines whether or not the switch 55a is in an ON state. The switch 55a is in the ON state when the instruction of outputting the high-frequency current is inputted by the user.

When it has been determined that the switch 55a is not in the ON state in the determination in step S10, the processor 55c proceeds to step S200. In step S200, the processor 55c determines whether or not the high-frequency power control device 55 is supplying electricity to the electrode unit 30.

When it has been determined that electricity is supplied to the electrode unit 30 in the determination in step S200, the processor 55c proceeds to step S210. In step S210, the processor 55c stops supply of electricity from the high-frequency power control device 55 to the electrode unit 30 and returns to step S10. When it has been determined that electricity is not supplied to the electrode unit 30 in the determination in step S200, the processor 55c returns to step S10. In other words, when the switch 55a is not in the ON state, the high-frequency power control device 55 does not supply electricity to the electrode unit 30.

On the other hand, when it has been determined that the switch 55a is in the ON state, the processor 55c executes processes of step S20 and steps thereafter.

In step S20, the processor 55c detects an electrical resistance value among the plurality of detection electrodes 39. More specifically, the processor 55c passes a minute current of a first output among the plurality of detection electrodes 39 and detects the resistance value of the current by means of the resistance detection unit 55b. The first output is smaller than a second output, which is an output of the high-frequency current to be passed to the treatment electrode 35 upon treatment (for example, resection or coagulation) of the tissue.

Next, in step S30, the processor 55c determines whether or not the resistance value detected by the resistance detection unit 55b is greater than or equal to a predetermined threshold value Th. The threshold value Th of the treatment is a resistance value slightly greater than a resistance value in the case in which the plurality of detection electrodes 39 are exposed into an electrically conductive perfusate. In the present embodiment, the electrically conductive perfusate is physiological saline.

When it has been determined that the resistance value is greater than or equal to the threshold value Th in the determination in step S30, the processor 55c proceeds to step S31. In step S31, the processor 55c detects pressure of the perfusate inside the organ 100 by means of the pressure sensor 12.

Next, in step S32, the processor 55c determines whether or not the pressure of the perfusate inside the organ 100 is within a predetermined appropriate range.

When it has been determined that the pressure of the perfusate inside the organ 100 is within the predetermined appropriate range in the determination in step S32, the processor 55c proceeds to step S40. In step S40, the processor 55c starts outputting the high-frequency current of the second output to the treatment electrode 35. As described above, the high-frequency current of the second output is to be outputted upon treatment (for example, resection or coagulation) of the tissue.

In other words, when the electrical resistance value among the plurality of detection electrodes 39 is greater than or equal to the predetermined threshold value, and the pressure of the perfusate inside the organ 100 is within the predetermined range, the processor 55c permits output of the high-frequency current of the second output to the treatment electrode 35.

Getting back to the description of the branch at step S30. When it has been determined that the resistance value is less than the threshold value Th in the determination in step S30, the processor 55c proceeds to step S50. In step S50, the processor 55c outputs information indicating an alert by means of the information output unit 55b. The information indicating an alert includes information notifying the user that the attitude of the electrode unit 30 is not appropriate. And then the processor 55c proceeds to step S210 and stops supply of electricity from the high-frequency power control device 55 to the electrode unit 30. In other words, when the electrical resistance value among the plurality of detection electrodes 39 is less than the predetermined threshold value, the processor 55c inhibits output of the high-frequency current of the second output to the treatment electrode 35.

Getting back to the description of the branch at step S32. When it has been determined that the pressure of the perfusate inside the organ 100 is out of the predetermined appropriate range in the determination in step S32, the processor 55c proceeds to step S33. In step S33, the processor 55c outputs information indicating an alert by means of the information output unit 55b. The information indicating an alert includes information notifying the user that the pressure of the perfusate inside the organ 100 is not appropriate. And then the processor 55c proceeds to step S210 and stops supply of electricity from the high-frequency power control device 55 to the electrode unit 30.

In other words, when the pressure of the perfusate inside the organ 100 is out of the appropriate range, the processor 55c inhibits output of the high-frequency current of the second output to the treatment electrode 35, regardless of magnitude of the electrical resistance value among the plurality of detection electrodes 39.

When the pressure of the perfusate filling the organ 100 changes, the thickness of the wall surface of the organ 100 changes. For example, as the pressure of the perfusate filling the organ 100 increases, the organ 100 swells, whereby the wall surface of the organ 100 is thinned. On the other hand, as the pressure of the perfusate filling the organ 100 decreases, the organ 100 contracts, whereby the wall surface of the organ 100 is thickened.

Even when the depth to which the treatment electrode 35 enters the tissue is constant, the change in the thickness of the wall surface of the organ 100 may change the thickness of the tissue to be resected by the treatment electrode 35. As described above, the endoscope system 1 according to the present embodiment can pass the high-frequency current from the treatment electrode 35 to resect the tissue only when the pressure of the perfusate filling the organ 100 is within the predetermined appropriate range. In other words, the endoscope system 1 according to the present embodiment can maintain the thickness of the tissue to be resected substantially constant, due to the configuration of resecting the tissue only when the thickness of the wall surface of the organ 100 is within a predetermined range.

### Fifth Embodiment

Hereinafter, the fifth embodiment of the present invention is described. In the following description, only differences from the first embodiment are explained, and constitutive elements similar to the constitutive elements of the first embodiment are denoted by the same reference symbols and description of these constitutive elements is omitted as appropriate.

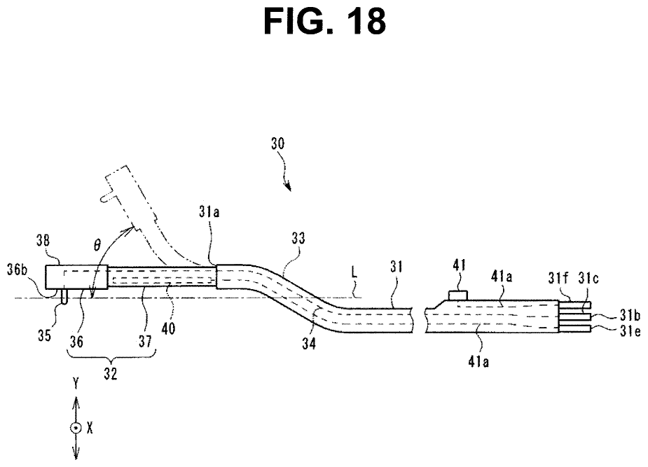

The electrode unit 30 of the present embodiment shown in FIG. 18 is provided with a bend detection sensor 40 and an attitude detection sensor 41 as the detection sensor, instead of the detection electrode 39 of the first embodiment.

The bend detection sensor 40 is a sensor configured to detect a bend amount θ of the elastic region 37. The bend detection sensor 40 is, for example, a strain sensor arranged in the elastic region 37. The bend detection sensor 40 is electrically connected to the detection connection portion 31e via a detection cable 40a inserted into the electrode unit 30. The processor 55c of the high-frequency power control device 55 can detect the bend amount θ of the elastic region 37 on the basis of a signal outputted from the bend detection sensor 40.

The bend amount θ of the elastic region 37 is represented by an amount of change in the angle of the distal end rigid portion 36 with respect to the proximal end rigid portion 31. More specifically, as shown in FIG. 18, the bend amount θ is an angle formed by the longitudinal axis L and the lower end face 36b when seen in the direction parallel to the first axis X. As shown by a solid line in FIG. 18, when the longitudinal axis L is parallel to the lower end face 36b, the bend amount θ is 0°. The value of the bend amount θ will increase when the elastic region 37 is bent while the lower end face 36b is directed outward as shown by a dashed-two dotted line in FIG. 18.

The attitude detection sensor 41 is a sensor configured to detect an inclination amount with respect to a gravity direction of the proximal end rigid portion 31. The attitude detection sensor 41 is, for example, an acceleration sensor arranged in the proximal end rigid portion 31. The attitude detection sensor 41 is electrically connected to a second detection connection portion 31f via a second detection cable 41a inserted into the proximal end rigid portion 31.

The second detection connection portion 31f is electrically connected to the detection connector 25 of the resectoscope 10 in a state in which the proximal end rigid portion 31 is fixed to the electrode unit holding portion 23. The processor 55c of the high-frequency power control device 55 can detect the inclination amount of the proximal end rigid portion 31 with respect to the gravity direction on the basis of a signal outputted from the attitude detection sensor 41.

As used herein, the inclination amount is an angle formed by the longitudinal axis L and the gravity direction. When the distal end 31a of the proximal end rigid portion 31 is positioned in the gravity direction (on the lower side) relative to the proximal end 31b, and the longitudinal axis L is vertical, the inclination amount ϕ is 0°. When the longitudinal axis L is horizontal, the inclination amount ϕ is 90°.

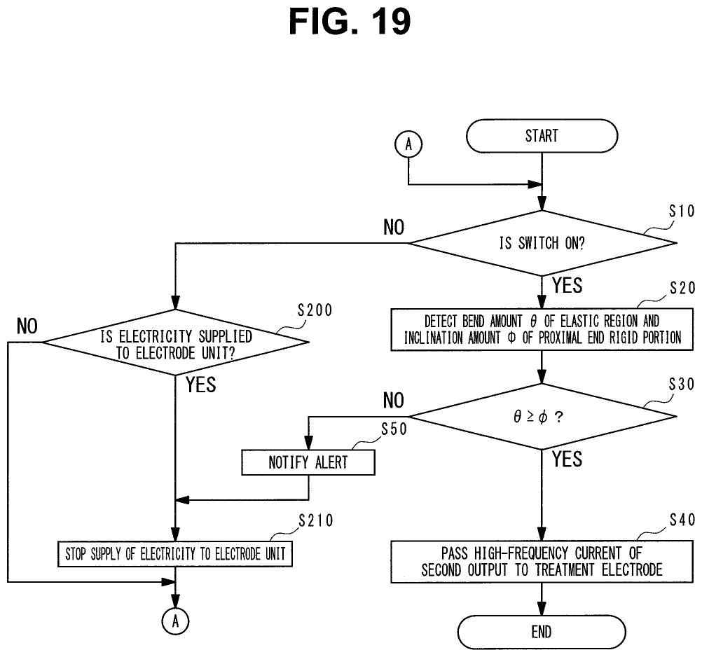

FIG. 19 is a flowchart showing an operation of the high-frequency power control device 55 of the present embodiment. The high-frequency power control device 55 of the present embodiment is different from the first embodiment in the operations in step S20 and step S30.

In the present embodiment, in step S20, the processor 55c detects the bend amount θ of the elastic region 37 and the inclination amount of the proximal end rigid portion 31 on the basis of signals outputted from the bend detection sensor 40 and the attitude detection sensor 41.

Then, in step S30, the processor 55c determines whether or not the bend amount θ of the elastic region 37 is greater than or equal to the inclination amount of the proximal end rigid portion 31. In other words, in the present embodiment, the processor 55c sets the inclination amount as a threshold value, and determines whether or not the bend amount θ is greater than or equal to the threshold value.

When it has been determined that the bend amount θ of the elastic region 37 is greater than or equal to the inclination amount of the proximal end rigid portion 31 in the determination in step S30, the processor 55c proceeds to step S40. On the other hand, when it has been determined that the bend amount θ of the elastic region 37 is less than the inclination amount of the proximal end rigid portion 31 in the determination in step S30, the processor 55c proceeds to step S50.

As described above, the processor 55c of the present embodiment permits output of the high-frequency current of the second output to the treatment electrode 35 when the bend amount θ of the elastic region 37 is greater than or equal to the inclination amount of the proximal end rigid portion 31, and inhibits output of the high-frequency current of the second output to the treatment electrode 35 when the bend amount θ of the elastic region 37 is less than the inclination amount of the proximal end rigid portion 31.

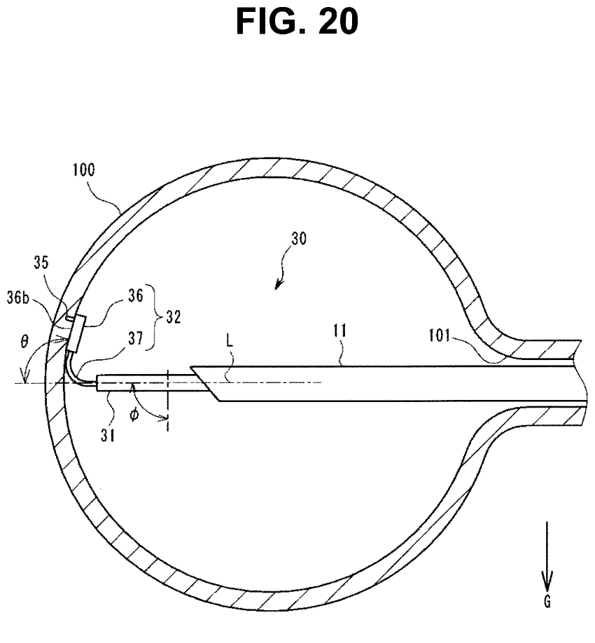

A case of resecting tissue in the organ 100, which is the bladder of the subject, by using the electrode unit 30 and the endoscope system 1 according to the present embodiment is described. For example, as shown in FIG. 20, in a case of resecting tissue of a wall surface in the bladder (organ 100) facing the urethral opening 101, the proximal end rigid portion 31 is substantially horizontal and the inclination amount is substantially 90°. Note that, in FIG. 20, a direction toward a lower side of the diagram is the gravity direction G. In this case, when the bend amount θ of the elastic region 37 is greater than or equal to 90°, it is highly likely that the bend amount of the elastic region 37 is sufficient as shown in FIG. 20 and the lower end face 36b of the distal end rigid portion 36 is entirely in contact with the tissue.

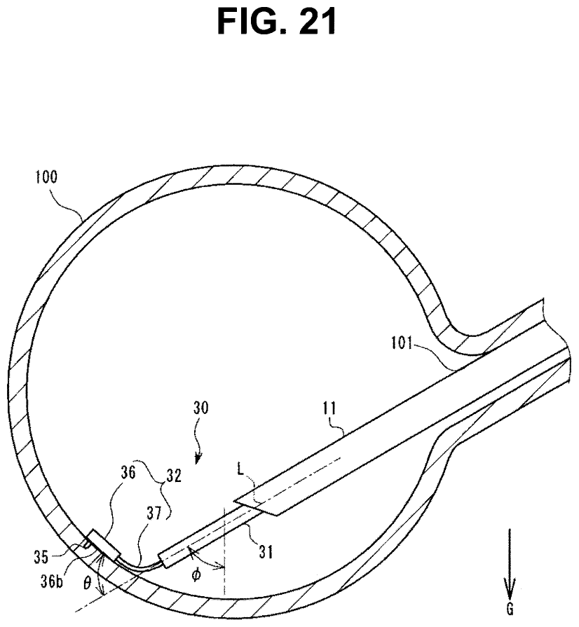

Alternatively, for example, as shown in FIG. 21, in a case of resecting tissue of a wall surface in the bladder (organ 100) positioned in the gravity direction (on the lower side) relative to the urethral opening 101, the proximal end rigid portion 31 forms an angle less than 90°. Note that, in FIG. 21, a direction toward a lower side of the diagram is the gravity direction G. In the case in which the inclination amount ϕ is 60° as shown in FIG. 21, when the bend amount of the elastic region 37 is greater than or equal to 60°, it is highly likely that the bend amount of the elastic region 37 is sufficient and the lower end face 36b of the distal end rigid portion 36 is entirely in contact with the tissue.

As explained in the foregoing, in the case in which the attitude of the treatment electrode 35 with respect to the tissue is different from the desired state, the electrode unit 30 and the endoscope system 1 according to the present embodiment stop outputting the high-frequency current from the high-frequency power control device 55. In other words, by using the electrode unit 30 and the endoscope system 1 according to the present embodiment, the depth to which the treatment electrode 35 enters the tissue can be maintained constant during a period of carrying out resection of the tissue by passing the high-frequency current from the treatment electrode 35.

Note that the configuration for detecting the bend amount θ of the elastic region 37 is not limited to the present embodiment. For example, it may also be configured to provide in the distal end rigid portion 36 a second attitude detection sensor configured to detect an inclination of the distal end rigid portion 36 with respect to the gravity direction, and to detect the bend amount θ on the basis of a comparison between a detection result of the second attitude detection sensor and a detection result of the attitude detection sensor 41.

Although in the present embodiment, the value of the inclination amount detected by using the attitude detection sensor 41 is used as is as the threshold value for the determination in step S30, the threshold value used in step S30 may also be a value obtained by multiplying the inclination amount by a coefficient or the like.

Although in the present embodiment, the attitude detection sensor 41 is provided in the proximal end rigid portion 31, the attitude detection sensor 41 may also be provided in the sheath 11, the slider 20, the telescope 21 or the like of the resectoscope 10.

### Sixth Embodiment

Hereinafter, the fifth embodiment of the present invention is described. In the following description, only differences from the fifth embodiment are explained, and constitutive elements similar to the constitutive elements of the first embodiment are denoted by the same reference symbols and description of these constitutive elements is omitted as appropriate.

Although the endoscope system 1 according to the fifth embodiment is provided with a sensor for the processor 55c of the high-frequency power control device 55 to automatically detect the bend amount θ, the endoscope system 1 according to the present embodiment is provided with a plurality of indices for the user to visually recognize the bend amount θ.

The endoscope system 1 is provided with first to fourth indices visually recognizable by the user. A first index 42, a second index 43, and a third index 44 are provided in the electrode unit 30. In addition, a fourth index 45 is an image generated by the high-frequency power control device 55 and displayed on the image display device 53.

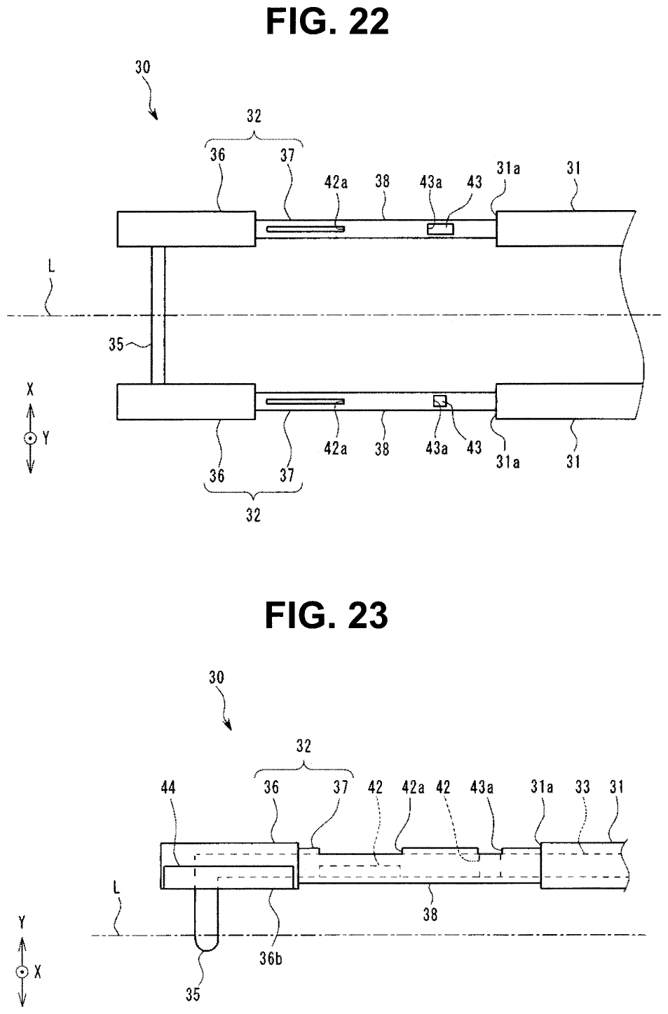

As shown in FIG. 23, the first index 42 is provided on the surface of a portion of the wire 33 inserted into the elastic region 37. More specifically, as shown in FIG. 22, an L-direction slit 42a, which is an opening penetrating from an upper face of the covering portion 38 to the wire 33, is formed in the elastic region 37. The L-direction slit 42a is an elongated through hole the longitudinal direction of which corresponds to a direction along the longitudinal axis L. The first index 42 is obtained by coloring the lower face of the wire 33 in a position where the L-direction slit 42a is formed.

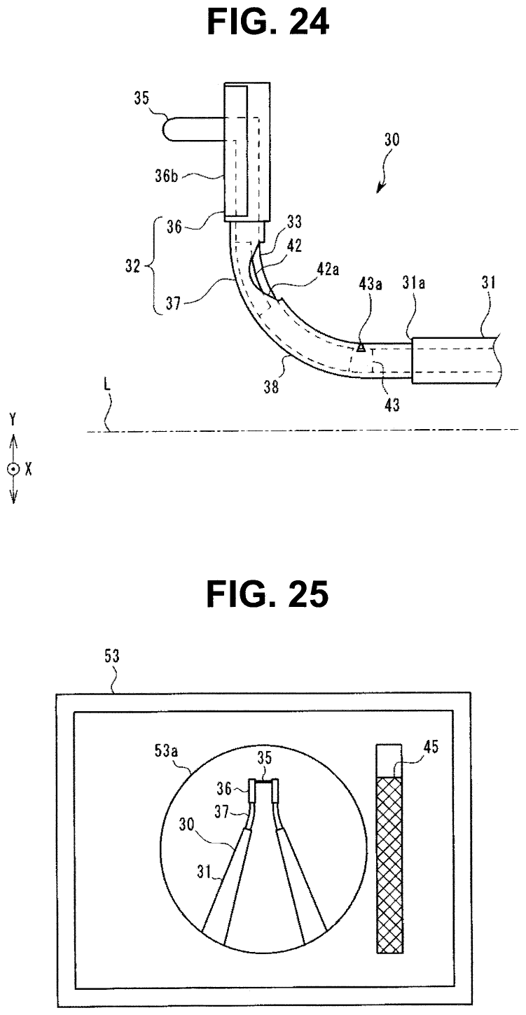

When the elastic region 37 is in a linear shape as shown in FIG. 23, the first index 42 is covered by the covering portion 38 and not exposed to the outside. When the elastic region 37 is curved with the upper face directed inward as shown in FIG. 24, the opening width of the L-direction slit 42a increases in the lateral direction, whereby the first index 42 is exposed to the outside. By means of an image picked up by using the telescope 21, the user can visually recognize the first index 42 exposed to the outside.

In the present embodiment, as shown in FIG. 22, the L-direction slit 42a and the first index 42 are provided on each of the pair of elastic regions 37. And then, in the L-direction slit 42a and the first index 42 provided on the right-side elastic region 37, the first index 42 is exposed to the outside when the bend amount θ of the elastic region 37 is greater than or equal to 90°. On the other hand, in the L-direction slit 42a and the first index 42 provided on the left-side elastic region 37, the first index 42 is exposed to the outside when the bend amount θ of the elastic region 37 is greater than or equal to 60°.

As shown in FIG. 23, the second index 43 is provided on the surface of a portion of the wire 33 inserted into the elastic region 37. More specifically, as shown in FIG. 22, a radial slit 43a, which is an opening penetrating from an upper face of the covering portion 38 to the wire 33, is formed in the elastic region 37. The radial slit 43a is an elongated through hole the longitudinal direction of which corresponds to a direction orthogonal to the longitudinal axis L. The second index 43 is obtained by coloring the outer peripheral face of the wire 33 in a position where the radial slit 43a is formed.

When the elastic region 37 is in a linear shape as shown in FIG. 23, the second index 42 is exposed to the outside through the radial slit 43a. In this case, by means of an image picked up by using the telescope 21, the user can visually recognize the second index 43 exposed to the outside. When the elastic region 37 is curved with the upper face directed inward as shown in FIG. 24, the opening width of the radial slit 43a decreases, whereby the second index 42 is not exposed to the outside.

In the present embodiment, as shown in FIG. 22, the radial slit 43a and the second index 43 are provided on each of the pair of elastic regions 37. And then, in the radial slit 43a and the second index 43 provided on the right-side elastic region 37, the second index 43 is not exposed to the outside when the bend amount θ of the elastic region 37 is greater than or equal to 90°. On the other hand, in the radial slit 43a and the second index 43 provided on the left-side elastic region 37, the first index 42 is not exposed to the outside when the bend amount θ of the elastic region 37 is greater than or equal to 60°.

The user can recognize the bend amount θ of the elastic region 37 on the basis of whether or not the first index 42 and the second index 43 are visually recognizable in the image picked up by the telescope 21 and displayed on the image display device 53.

As shown in FIG. 23, the third index 44 is obtained by coloring the lower face of the distal end rigid portion 36. When the lower end face 36b of the distal end rigid portion 36 is entirely in contact with the wall surface of the organ 100, the third index 44 is covered by the tissue of the organ 100.

The user can recognize a manner in which the lower end face 36b of the distal end rigid portion 36 is in contact with the wall surface, on the basis of whether or not the third index 44 is visually recognizable in the image picked up by the telescope 21 and displayed on the image display device 53.

The fourth index 45 is, as shown in FIG. 25, a part of the image generated by the high-frequency power control device 55 and displayed on the image display device 53. The fourth index 45 is arranged beside an endoscope image 53a picked up by using the telescope 21. The fourth index 45 indicates an upper limit of the appropriate position of the distal end of the electrode unit 30 in a vertical direction in the endoscope image 53a.

The resectoscope 10 according to the present embodiment is provided with a slider operation amount detection unit configured to detect the position of the slider 20. The processor 55c of the high-frequency power control device 55 calculates the bend amount θ (appropriate bend amount) of the elastic region 37 required to bring the lower end face 36b of the distal end rigid portion 36 entirely into contact with the tissue, on the basis of the inclination amount detected by using the attitude detection sensor 41. And then, the processor 55c calculates the position of the distal end of the electrode unit 30, in the endoscope image 53a, that is appropriate for bringing the lower end face 36b of the distal end rigid portion 36 entirely into contact with the tissue, on the basis of the appropriate bend amount and the position of the slider 20. Thereafter, the processor 55c displays on the image display device 53, the appropriate position of the distal end of the electrode unit 30 thus calculated as the fourth index 45.

The user can recognize that the bend amount θ of the elastic region 37 is sufficient when the position of the distal end of the electrode unit 30 is on the upper side relative to the fourth index 45 in the endoscope image 53a on the image display device 53.

As explained in the foregoing, according to the electrode unit 30 and the endoscope system 1 according to the present embodiment, the user can easily recognize the bend amount θ of the elastic region 37 on the basis of visual perception, and can bring the lower end face 36b of the distal end rigid portion 36 entirely in contact with the tissue.

The present invention is not limited to the aforementioned embodiments and can be modified as appropriate without departing from the gist or spirit of the present invention that can be read from the claims and the specification as a whole. An electrode unit and an endoscope system with such a modification are also encompassed in the technical scope of the present invention.

What is claimed is:

1. An electrode unit configured to resect or coagulate tissue inside a subject by using a high-frequency current, comprising:
an electrode supporting member provided with a pair of distal end rigid members surfaces of which are covered by an electrically insulating material;
a treatment electrode configured with electrode bodies arranged in respective distal end portions of the pair of distal end rigid members and protruding downward respectively from the pair of distal end rigid members, and a spanning portion that spans respective lower end portions of the electrode bodies; and
a detection sensor arranged on respective proximal end sides of the pair of distal end rigid members and configured to detect a contact state between the pair of distal end rigid members and the tissue.

2. An electrode unit configured to resect or coagulate tissue inside a subject by using a high-frequency current, comprising:
an electrode supporting member provided with a pair of distal end rigid members surfaces of which are covered by an electrically insulating material, and an elastic member rigidity of which is lower than rigidity of the pair of distal end rigid members;
a treatment electrode configured with electrode bodies arranged in respective distal end portions of the pair of distal end rigid members and protruding downward respectively from the pair of distal end rigid members, and a spanning portion that spans respective lower end portions of the electrode bodies;
a proximal end rigid member arranged on a proximal end side of the elastic member;
a bend detection sensor configured to detect a bend amount of the elastic member; and
an attitude detection sensor configured to detect an inclination amount with respect to a gravity direction of the pair of distal end rigid members.

3. An electrode unit configured to treat tissue inside a subject by using a high-frequency current under observation with an endoscope, comprising:
an electrode supporting member inserted into the subject, an external surface of which is configured with an electrically insulating material;
a distal end rigid member provided in a distal end portion of the electrode supporting member;
a treatment electrode supported by the distal end rigid member and protruding from an external surface of the distal end rigid member;
a collection electrode electrically connected with the treatment electrode; and
a detection electrode arranged on a proximal end side relative to the treatment electrode on a face of the external surface of the distal end rigid member, the face being oriented in a protrusion direction of the treatment electrode, the detection electrode being configured to detect a contact state between the distal end rigid member and the tissue.

4. An endoscope system comprising:
the electrode unit according to claim 1;
a high-frequency power control device configured to control output of high-frequency waves; and
a resistance detection circuit provided in the high-frequency power control device and configured to detect a resistance value of a current flowing in the electrode unit,
wherein in the high-frequency power control device, the detection sensor detects a resistance value of a current flowing between the pair of distal end rigid members, and an output of a high-frequency current to the treatment electrode is controlled on the basis of the resistance value.

5. The endoscope system according to claim 4, wherein:
the electrode unit is provided with the detection sensor in plurality;
the resistance detection circuit detects a resistance value among the detection sensor in plurality; and
the high-frequency power control device permits the output of the high-frequency current to the treatment electrode when the resistance value detected by the resistance detection circuit is greater than or equal to a predetermined threshold value, and inhibits the output of the high-frequency current to the treatment electrode when the resistance value is less than the predetermined threshold value.

6. The endoscope system according to claim 4 further comprising a collection electrode,
wherein:
the resistance detection circuit detects a resistance value between the detection sensor and the collection electrode; and
the high-frequency power control device permits the output of the high-frequency current to the treatment electrode when the resistance value detected by the resistance detection circuit is greater than or equal to a predetermined threshold value, and inhibits the output of the high-frequency current to the treatment electrode when the resistance value is less than the predetermined threshold value.

7. The endoscope system according to claim 5, further comprising a pressure sensor configured to detect a pressure of a perfusate in the subject,
wherein the high-frequency power control device inhibits the output of the high-frequency current to the treatment electrode regardless of the resistance value when the pressure of the perfusate detected by the pressure sensor is out of a predetermined range.

8. An endoscope system comprising:
the electrode unit according to claim 2;
a high-frequency power control device configured to control output of high-frequency waves; and
a processor provided in the high-frequency power control device,
wherein the processor detects an attitude of the distal end rigid member with respect to the tissue on the basis of the bend amount and the inclination amount, and controls the output of the high-frequency current to the treatment electrode on the basis of the attitude.

9. An endoscope system comprising:
the electrode unit according to claim 3;
a high-frequency power control device configured to control output of high-frequency waves; and
a resistance detection circuit provided in the high-frequency power control device and configured to detect a resistance value of a current flowing in the electrode unit,
wherein in the high-frequency power control device, the detection electrode detects a resistance value of a current flowing between the pair of distal end rigid members, and an output of a high-frequency current to the treatment electrode is controlled on the basis of the resistance value.

10. The endoscope system according to claim 9, wherein:
the electrode unit is provided with the detection electrode in plurality;
the resistance detection circuit detects a resistance value among the detection electrode in plurality; and
the high-frequency power control device permits the output of the high-frequency current to the treatment electrode when the resistance value detected by the resistance detection circuit is greater than or equal to a predetermined threshold value, and inhibits the output of the high-frequency current to the treatment electrode when the resistance value is less than the predetermined threshold value.

11. The endoscope system according to claim 9, wherein:
the resistance detection circuit detects a resistance value between the detection electrode and the collection electrode; and
the high-frequency power control device permits the output of the high-frequency current to the treatment electrode when the resistance value detected by the resistance detection circuit is greater than or equal to a predetermined threshold value, and inhibits the output of the high-frequency current to the treatment electrode when the resistance value is less than the predetermined threshold value.

12. The endoscope system according to claim 10, further comprising a pressure sensor configured to detect a pressure of a perfusate in the subject,
wherein the high-frequency power control device inhibits the output of the high-frequency current to the treatment electrode regardless of the resistance value when the pressure of the perfusate detected by the pressure sensor is out of a predetermined range.
